(19) United States
(12) Patent Application Publication
(10) Pub. No.: US 2023/0127884 A1
(43) Pub. Date: Apr. 27, 2023

(54) A PROCESS FOR THE PREPARATION OF 4-CYANOBENZOYL CHLORIDES

(71) Applicant: BASF SE, Ludwigshafen (DE)

(72) Inventors: Florian Vogt, Ludwigshafen (DE); Joachim Gebhardt, Ludwigshafen (DE); Kailaskumar Borate, Navi Mumbai (IN); Bernd Wolf, Ludwigshafen (DE); Heinz Friedrich Sutoris, Ludwigshafen (DE); Roland Goetz, Ludwigshafen (DE)

(21) Appl. No.: 17/918,137

(22) PCT Filed: Apr. 12, 2021
(86) PCT No.: PCT/EP2021/059424

§ 371 Date: Oct. 11, 2022

(30) Foreign Application Priority Data
Apr. 16, 2020 (IN) 202021016414
Jun. 16, 2020 (EP) 20180312.9

(51) Int. Cl. C07C 253/30; C07D 271/06

(52) U.S. Cl. CPC C07C 253/30; C07D 271/06

(57) ABSTRACT

The present invention relates to a process for the preparation of 4-cyanobenzoyl chlorides of formula I through reaction of compounds of formula II with a chlorinating agent.

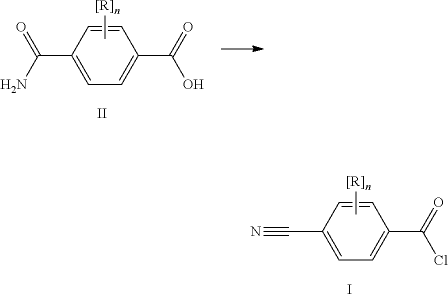

The present invention relates to a process for the preparation of 4-cyanobenzoyl chlorides of formula I through reaction of compounds of formula II with a chlorinating agent.

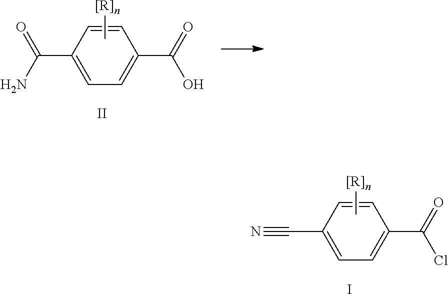

4-Cyanobenzoyl chlorides of formula I are versatile and highly reactive synthetic intermediates. The functional groups offer various strategic options for orthogonal synthesis concepts taking advantage of the differentiated reactivity of the nitrile and the carboxylic acid chloride group towards a large number of reactants/reagents. For example, compounds of formula I can be employed in the efficient preparation of known benzamide type trifluoromethyl-1,2,4-oxadiazoles, for example compounds disclosed in WO 2015/185485 A1 and WO 2017/211649 A1, which are useful for controlling phytopathogenic fungi.

Synthetic access to 4-cyanobenzoyl chlorides of formula I via 4-carbamoylbenzoic acids of type II is particularly attractive since it taps into industrial feedstock such as terephthalic acid dichloride, which is available on industrial scale at low price. For instance, desymmetrization of terephthalic acid dichloride affords compounds of formula II, wherein R is hydrogen, in two simple steps.

It is part of the common general knowledge of the skilled person in the art of synthetic chemistry that carboxylic acids react with suitable chlorinating agents to yield carboxylic acid chlorides. Likewise, it is widely known that primary carboxamides can undergo dehydration with the same type of chlorinating agents to obtain the corresponding nitriles. However, the prior art does not report selective transformations of compounds featuring both these functional groups in one molecule, and to transform each functional group in one step using one type of chlorinating agent.

The skilled person in the art knows that carboxylic acid halides are highly reactive electrophiles, which react non-selectively with nucleophilic species, for example with primary carboxamides. In the same sense the reaction of a primary carboxamide that eventually leads to the formation of a nitrile proceeds via highly reactive intermediate species that are prone to intermolecular reactions. Given these properties, the skilled person would expect a great number of side reactions when using chlorinating agents with bifunctional compounds of formula II, including, amongst others, the undesired formation of oligomers and/or polymers. Accordingly, the skilled person would not have expected any appreciable conversion of compounds II in favor of the desired compounds of formula I and he would not have considered to use them in large-scale processes for the preparation of 4-cyanobenzoyl chlorides of formula I.

The inventors surprisingly found that against all odds moderate to good yields of compounds I can be obtained by reaction of compounds II with chlorinating agents. This process provides an economic process enabling the efficient preparation of compounds of formula I on an industrial scale in high yield and with low amounts of side-products.

Accordingly, the present invention relates to a process for preparing 4-cyanobenzoyl chlorides of formula I,

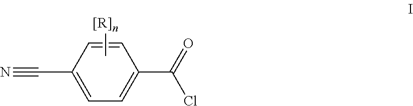

wherein

R is halogen, C₁-C₄-alkyl, C₁-C₄-haloalkyl, C₁-C₄-alkoxy, or C₁-C₄-haloalkoxy;

n is 0, 1 or 2;

the process comprising reacting a compound of formula II,

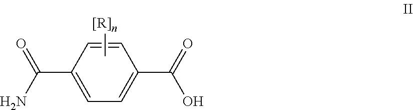

wherein the variable R is as defined above for compounds of formula I, with a chlorinating agent.

In one embodiment the chlorinating agent of the process of the invention is selected from the group consisting of phosphoryl trichloride, trichlorophosphane, pentachlorophosphane, thionyl chloride, phosgene, diphosgene, triphosgene, and oxalyl chloride; in a preferred embodiment the chlorinating agent is phosphoryl trichloride.

Typically, the amount of the chlorinating agent is between 2 and 15 equivalents, preferably between 2 and 10 equivalents, more preferably between 2 and 8 equivalents, based on the amount of compound II.

The process proceeds even faster in the presence of small amounts of N,N-dimethylformamide or N,N-dimethylacetamide. Therefore, in one aspect of the present invention the process is conducted in the presence of substoichiometric amounts of N,N-dimethylformamide or N,N-dimethylacetamide, preferably N,N-dimethylformamide, based on the amount of compound II. In one aspect N,N-dimethylformamide or N,N-dimethylacetamide, preferably N,N-dimethylformamide, is used in an amount of up to 0.5 equivalents, up to 0.2 equivalents, or up to 0.1 equivalents, based on the amount of compound II. In another aspect N,N-dimethylformamide or N,N-dimethylacetamide, preferably N,N-dimethylformamide, is used in an amount that is in the range between 0.01 and 0.5 equivalents, 0.05 and 0.2 equivalents, or 0.05 and 0.1 equivalents, based on the amount of compound II.

The process of the present invention is conducted either in an auxiliary solvent, or in the absence of an auxiliary solvent. The term “auxiliary solvent” herein refers to an inert aprotic organic solvent, which acts merely as a solvent and is not consumed in the course of the reaction. For the avoidance of doubt an auxiliary solvent is not identical with the reactants such as compounds II, the chlorinating agent, N,N-dimethylformamide or N,N-dimethylacetamide.

Suitable auxiliary solvents are, for example, aliphatic, cycloaliphatic and aromatic hydrocarbons (non-limiting examples are: pentane, hexane, petroleum ether, cyclohexane, methylcyclohexane, benzene, toluene, xylene), aliphatic halogen-hydrocarbons (non-limiting examples are: methylene chloride, chloroform, di- and tetrachloroethane), nitriles (non-limiting examples are: acetonitrile, propionitrile, benzonitrile), ethers (non-limiting examples are: diethylether, dibutylether, tert-butylmethylether, ethylene glycol dimethyl ether, ethylene glycol, diethyl ether, diethylene glycol dimethyl ether, dioxane, diethylene, glycol monomethyl- or monoethyl ether), and sulphoxides and sulphones (non-limiting examples are: dimethyl sulfoxide, dimethyl sulfone, tetramethylene sulfoxide, tetramethylene sulfone).

Preferred auxiliary solvents are dioxane, tert-butyl methyl ether, di-iso-propyl ether, benzene, toluene, xylene, mesitylene, chlorobenzene, n-hexane, cyclohexane, dichloromethane, chloroform, tetrachloromethane, dichloroethane, or mixtures thereof.

In another preferred aspect the process is conducted in the absence of an auxiliary solvent.

In one embodiment the process is conducted at a concentration of at least 10% by weight of compound II, based on the total reaction medium. In another preferred embodiment the oxidation process is conducted at a concentration of at least 15% by weight of compound II, based on the total reaction medium.

The reaction mixture in these processes, with or without an auxiliary solvent, is usually heated to reflux temperature or to a temperature that is within the range between the reflux temperature and a temperature that lies 50° C. below the reflux temperature; preferably the reaction mixture is heated to a temperature that is within the range between the reflux temperature and a temperature that lies 30° C. below the reflux temperature; more preferably the reaction mixture is heated to a temperature that is within the range between the reflux temperature and a temperature that lies 10° C. below the reflux temperature. In a particularly preferred embodiment the process of the present invention the reaction mixture is heated at reflux.

The reaction is carried out at pressures within a range between 100 kPa (1 bar) and 500 kPa, preferably between 100 kPa and 300 kPa.

The reaction is generally carried out within 1 to 12 hours; preferably within 1 to 8 hours; more preferably within 1 to 6 hours. Even more preferred is a reaction time within 1 to 4 hours.

In one aspect of the present invention the variable n is 1 and R is fluorine.

In a preferred embodiment the variable n is 0.

In a preferred embodiment (embodiment E.1) of the present invention the chlorinating reagent is phosphoryl trichloride.

Embodiment E.2: is based on embodiment E.1, wherein the amount of the chlorinating agent is between 2 and 15 equivalents.

Embodiment E.3: is based on embodiment E.2, wherein the auxiliary solvent is dioxane, tert-butyl methyl ether, di-iso-propyl ether, benzene, toluene, xylene, mesitylene, chlorobenzene, n-hexane, cyclohexane, dichloromethane, chloroform, tetrachloromethane, dichloroethane, or mixtures thereof; or in the absence of an auxiliary solvent.

Embodiment E.4: is based on embodiment E.3, wherein the reaction mixture is heated to a temperature that is within the range between the reflux temperature and a temperature that lies 50° C. below the reflux temperature.

Embodiment E.5: is based on embodiment E.3, wherein the reaction mixture is heated to a temperature that is within the range between the reflux temperature and a temperature that lies 30° C. below the reflux temperature.

Embodiment E.6: is based on embodiment E.3, wherein the reaction mixture is heated to a temperature that is within the range between the reflux temperature and a temperature that lies 10° C. below the reflux temperature.

Embodiment E.7: is based on embodiment E.4, wherein the pressure is within a range between 100 kPa and 500 kPa.

Embodiment E.8: is based on embodiment E.5, wherein the pressure is within a range between 100 kPa and 500 kPa.

Embodiment E.9: is based on embodiment E.6, wherein the pressure is within a range between 100 kPa and 500 kPa.

Embodiment E.10: is based on embodiment E.4, wherein the pressure is within a range between 100 kPa and 300 kPa.

Embodiment E.11: is based on embodiment E.5, wherein the pressure is within a range between 100 kPa and 300 kPa.

Embodiment E.12: is based on embodiment E.6, wherein the pressure is within a range between 100 kPa and 300 kPa.

Compounds of formula II can be obtained in a two-step process, which involves reacting a compound of formula IIa

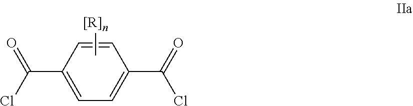

in the presence of water as described in Journal of Organic Chemistry 1953, 18, 1664-1669 to give a compound of formula IIb

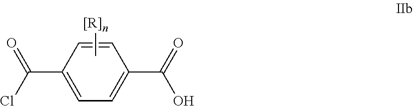

which is further reacted with ammonia as described in Journal of Organic Chemistry 2016, 81(5), 2166-2173 to give a compound of formula II, whereas the variables n and R in compounds IIa and IIb are as defined for compounds of formulae I and II herein.

The dichlorides IIa are either commercially available or they can be prepared from commercially available starting materials using synthetic procedures that are well known to the skilled person in the art.

In a further embodiment the present invention relates to a process comprising the step of reacting the compound of formula I, wherein the variable n is 0, with an amine of formula III,

R¹—NH—R² III

wherein
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, C₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂—C₁-C₄-alkyl, —(C═O)—C₁-C₄-alkyl, C(═O)—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)H, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;

to obtain a compound of formula IV

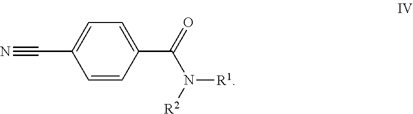

Analogous transformations are described in WO 2013/008162 A1, WO 2015/185485 A1, or WO 2017/211652 A1 and the references cited therein.

The amines of formula III are either commercially available or can be prepared, for example, according to R. C. Larock, Comprehensive Organic Transformations, Verlag Wiley-VCH, 2^{nd} Edition 1999, pages 1929 ﬀ.

In a further embodiment the present invention relates to a process comprising the step of reacting the compound of formula IV with hydroxylamine or its hydrochloride salt, in the presence of a base, preferably triethylamine, sodium hydroxide or sodium methylate, in a suitable solvent, such as methanol, ethanol or water, or a mixture of these solvents, at a temperature between 0° C. and 100° C. to obtain a compound of formula Va

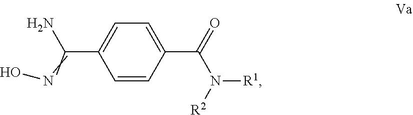

which is further reacted with an activated derivative of trifluoroacetic acid, for example ethyl trifluoroacetate, trifluoroacetic anhydride or trifluoroacetic chloride, to obtain a compound of formula V

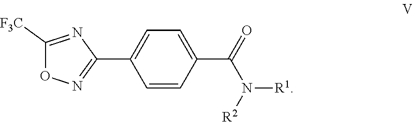

For related examples see Kitamura, S. et al Chem. Pharm. Bull. 2001, 49, 268 or WO 2013/008162 A1 or WO 2015/185485 A1.

In another embodiment, the compound of formula V is reacted with a suitable thionylating reagent to obtain a compound of formula VI

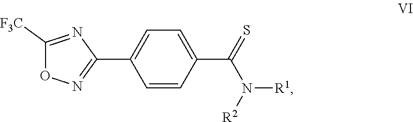

as described in WO 2019/020451 A1 and WO 2017/211649 A1 and the references cited therein.

In a preferred embodiment the variables R¹ and R² in compounds of formula III, IV, V and VI have the following meaning:
R¹ is methyl, ethyl, n-propyl, iso-propyl, n-butyl, sec-butyl, iso-butyl, cyclopropyl, 2-methoxyiminoethyl, bicyclo[1.1.1]pentan-1-yl, or phenyl; and wherein the phenyl group is unsubstituted or substituted with 1, 2, 3 or up to the maximum possible number of identical or different radicals selected from the group consisting of fluorine, chlorine, cyano, OH, NH₂, methyl, ethyl, methoxy, trifluoromethyl, trifluoromethoxy, difluoromethyl, difluoromethoxy, and cyclopropyl; and
R² is hydrogen, methyl, or ethyl.

In another preferred embodiment the variables R¹ and R² in compounds of formula III, IV, V and VI have the following meaning:
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl; and
R² is hydrogen.

In the definitions of the variables given above, collective terms are used which are generally representative for the substituents in question.

The term “Cₙ-Cₘ” indicates the number of carbon atoms possible in each case in the substituent or substituent moiety in question.

The term “halogen” refers to fluorine, chlorine, bromine and iodine.

The term “oxo” refers to an oxygen atom ═O, which is bound to a carbon atom or sulfur atom, thus forming, for example, a ketonyl —C(═O)— or sulfinyl —S(═O)— group.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₁-C₆-haloalkoxy” refers to a C₁-C₆-alkoxy group as defined above, wherein some or all of the hydrogen atoms may be replaced by halogen atoms as mentioned above, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy.

The terms “phenyl-C₁-C₄-alkyl or heteroaryl-C₁-C₄-alkyl” refer to alkyl having 1 to 4 carbon atoms (as defined above), wherein one hydrogen atom of the alkyl radical is replaced by a phenyl or heteroaryl radical respectively.

The term “C₁-C₄-alkoxy-C₁-C₄-alkyl” refers to alkyl having 1 to 4 carbon atoms (as defined above), wherein one hydrogen atom of the alkyl radical is replaced by a C₁-C₄-alkoxy group (as defined above). Likewise, the term “C₁-C₄-alkylthio-C₁-C₄-alkyl” refers to alkyl having 1 to 4 carbon atoms (as defined above), wherein one hydrogen atom of the alkyl radical is replaced by a C₁-C₄-alkylthio group.

The term “C₁-C₆-alkylthio” as used herein refers to straight-chain or branched alkyl groups having 1 to 6 carbon atoms (as defined above) bonded via a sulfur atom. Accordingly, the term “C₁-C₆-haloalkylthio” as used herein refers to straight-chain or branched haloalkyl group having 1 to 6 carbon atoms (as defined above) bonded through a sulfur atom, at any position in the haloalkyl group.

The term “C₁-C₄-alkoxyimino” refers to a divalent imino radical (C₁-C₄-alkyl-O—N═) carrying one C₁-C₄-alkoxy group as substituent, e.g. methylimino, ethylimino, propylimino, 1-methylethyl-imino, butylimino, 1-methylpropylimino, 2-methylpropylimino, 1,1-dimethylethylimino and the like.

The term “C₁-C₆-alkoxyimino-C₁-C₄-alkyl” refers to alkyl having 1 to 4 carbon atoms, wherein two hydrogen atoms of one carbon atom of the alkyl radical are replaced by a divalent C₁-C₆-alkoxyimino radical (C₁-C₆-alkyl-O—N═) as defined above.

The term “C₂-C₆-alkenyloxyimino-C₁-C₄-alkyl” refers to alkyl having 1 to 4 carbon atoms, wherein two hydrogen atoms of one carbon atom of the alkyl radical are replaced by a divalent C₂-C₆-alkenyloxyimino radical (C₂-C₆-alkenyl-O—N═).

The term “C₂-C₆-alkynyloxyimino-C₁-C₄-alkyl” refers to alkyl having 1 to 4 carbon atoms, wherein two hydrogen atoms of one carbon atom of the alkyl radical are replaced by a divalent C₂-C₆-alkynyloxyimino radical (C₂-C₆-alkynyl-O—N═).

The term “hydroxyC₁-C₄-alkyl” refers to alkyl having 1 to 4 carbon atoms, wherein one hydrogen atom of the alkyl radical is replaced by a OH group.

The term “aminoC₁-C₄-alkyl” refers to alkyl having 1 to 4 carbon atoms, wherein one hydrogen atom of the alkyl radical is replaced by a NH₂ group.

The term “C₁-C₆-alkylamino” refers to an amino group, which is substituted with one residue independently selected from the group that is defined by the term C₁-C₆-alkyl. Likewise, the term “diC₁-C₆-alkylamino” refers to an amino group, which is substituted with two residues independently selected from the group that is defined by the term C₁-C₆-alkyl.

The term “C₁-C₄-alkylamino-C₁-C₄-alkyl” refers to refers to alkyl having 1 to 4 carbon atoms (as defined above), wherein one hydrogen atom of the alkyl radical is replaced by a C₁-C₄-alkyl-NH-group which is bound through the nitrogen. Likewise, the term “diC₁-C₄-alkylamino-C₁-C₄-alkyl” refers to refers to alkyl having 1 to 4 carbon atoms (as defined above), wherein one hydrogen atom of the alkyl radical is replaced by a (C₁-C₄-alkyl)₂N— group which is bound through the nitrogen.

The term “aminocarbonyl-C₁-C₄-alkyl” refers to alkyl having 1 to 4 carbon atoms, wherein one hydrogen atom of the alkyl radical is replaced by a —(C═O)—NH₂ group.

The term “C₃-C₁₁-cycloalkyl” refers to a monocyclic, bicyclic or tricyclic saturated univalent hydrocarbon radical having 3 to 11 carbon ring members that is connected through one of the ring carbon atoms by substitution of one hydrogen atom, such as cyclopropyl (C₃H₅), cyclobutyl, cyclopentyl, cyclohexyl, cycloheptyl, cyclooctyl, bicyclo[1.1.0]butyl, bicyclo[2.1.0]pentyl, bicyclo[1.1.1]pentyl, bicyclo[3.1.0]hexyl, bicyclo[2.1.1]hexyl, norcaranyl (bicyclo[4.1.0]heptyl) and norbornyl (bicyclo[2.2.1]heptyl).

The terms “—C(═O)—C₁-C₆-alkyl”, “—C(═O)—O—C₁-C₆-alkyl” and “—C(═O)—C₃-C₁₁-cycloalkyl” refer to aliphatic radicals which are attached through the carbon atom of the —C(═O)— group.

The term “aliphatic” refers to compounds or radicals composed of carbon and hydrogen and which are non-aromatic compounds. An “alicyclic” compound or radical is an organic compound that is both aliphatic and cyclic. They contain one or more all-carbon rings which may be either saturated or unsaturated, but do not have aromatic character.

The terms “cyclic moiety” or “cyclic group” refer to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, C₃-C₈-cycloalkyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring.

The term “heteroaryl” refers to aromatic monocyclic or polycyclic ring systems including besides carbon atoms, 1, 2, 3 or 4 heteroatoms independently selected from the group consisting of N, O and S.

The term “saturated 3- to 7-membered carbocycle” is to be understood as meaning monocyclic saturated carbocycles having 3, 4 or 5 carbon ring members. Examples include cyclopropyl, cyclopentyl, cyclohexyl, cycloheptyl, and the like.

The term “3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms”, is to be understood as meaning both, aromatic mono- and bicyclic heteroaromatic ring systems, and also saturated and partially unsaturated heterocycles, for example:

a 3- or 4-membered saturated heterocycle which contains 1 or 2 heteroatoms from the group consisting of N, O and S as ring members such as oxirane, aziridine, thiirane, oxetane, azetidine, thiethane, [1,2]dioxetane, [1,2]dithietane, [1,2]diazetidine;

and a 5- or 6-membered saturated or partially unsaturated heterocycle which contains 1, 2 or 3 heteroatoms from the group consisting of N, O and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and

a 7-membered saturated or partially unsaturated heterocycle such as tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or 7-yl, 2,3,4,7-tetrahydro[1H]azepin-1-, -2-, -3-, -4-, -5-, or 7-yl, 2,3,6,7-tetrahydro[1H]azepin-1-, -2-, -3-, -4-, -5-, -6- or 7-yl, hexahydroazepin-1-, -2-, -3- or 4-yl, tetra- and hexahydroazepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or 7-yl, hexahydroazepin-1-, -2-, -3- or 4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “5- or 6-membered heteroaryl” or the term “5- or 6-membered aromatic heterocycle” refer to aromatic ring systems including besides carbon atoms, 1, 2, 3 or 4 heteroatoms independently selected from the group consisting of N, O and S, for example, a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or

a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

### WORKING EXAMPLES

The present invention is further illustrated by means of the following working examples.

HPLC Methodology

HPLC device: Agilent 1100 Series; column: Agilent Zorbax Eclipse XDB-C18 1.8 μm 50*4.6 mm von Agilent, Column Flow: 1.3 mL/min, time: 10 min, pressure: 23000 kPa; temperature: 20° C.; wavelength 195 nm; injector volume: 1 uL; retention time of the respective products is based on reference material and given below. Eluent: A: Water with 0.1 vol % H₃PO₄; B: Acetonitrile

| Time (min) | % B | Rate (mL/min) |
|---|---|---|
| 0.0 | 0 | 1.3 |
| 2.0 | 0 | 1.3 |
| 5.0 | 80 | 1.3 |
| 6.0 | 100 | 1.3 |
| 8.0 | 100 | 1.3 |
| 8.1 | 0 | 1.3 |

### Example 1) Preparation of 4-carbamoylbenzoic acid

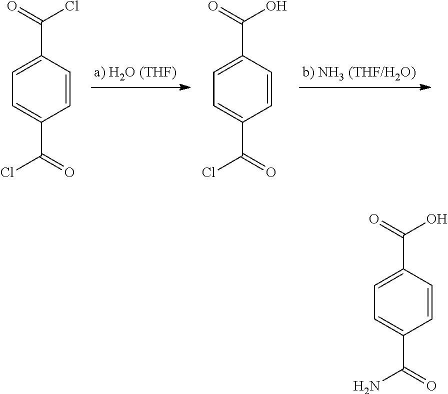 [SMILES: O=C(Cl)c1ccc(C(=O)Cl)cc1 ; O=C(O)c1ccc(C(=O)Cl)cc1 ; NC(=O)c1ccc(C(=O)O)cc1]

Step a): a three necked flask was charged with tetrahydrofuran (10 mL) and terephthalic acid dichloride (2.4 g, 11.8 mmol, commercially available). A solution of water (224 mg, 12.4 mmol, 1.05 eq.) in tetrahydrofuran (10 mL) was added dropwise and the resulting mixture was stirred at ambient temperature for 2 hours (for reaction control: an aliquot is quenched with methanol and the resulting mixture is analyzed via HPLC).

Step b): the crude product was transferred to a dropping funnel and added dropwise to a mixture of ammonia (33% w/w in water, 3.8 g, 35.4 mmol) and tetrahydrofuran (12 mL) over a period of 25 minutes. The resulting suspension was stirred for 1 hour at ambient temperature (for reaction control: an aliquot is quenched with methanol and the resulting mixture is analyzed via HPLC), water was added and the mixture was stirred for additional 30 minutes. An aqueous solution of hydrogen chloride (37% w/w in water) was added to adjust the pH value to 1 and stirring was continued for 30 minutes. The solids were collected by filtration, washed with water (2×10 mL) and dried under vacuum at 40° C. HPLC analysis showed 68 area % of 4-carbamoylbenzoic acid (retention time 4.90 min) along with 19% diamide, 11% diacid, 2% others.

Similar Experiments were Conducted with Varying Water Amounts:

The use of 0.9 eq. water with otherwise unchanged conditions delivers a mixture of:

monoacid—71 area % (retention time 4.90 min), diacid—9 area %, diamide—18 area %.

The use of 1.2 eq. water with otherwise unchanged conditions delivers a mixture of:

monoacid—71 area % (retention time 4.90 min), diacid—18 area %, diamide—8 area %.

### Example 2) Preparation of 4-cyanobenzoyl chloride

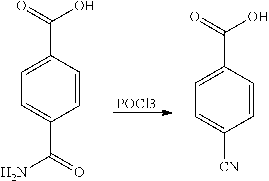

A mixture (8 g) obtained by the procedure of Example 1) containing the mono-acid (72.1 area %), the di-acid (4.9 area %) and the diamide (13.8 area %) was stirred with phosphoryl trichloride (52 g, 339 mmol) for 1 hour at an internal temperature of 80° C. During this time the solids dissolve completely in the chlorinating reagent. An aliquot is carefully quenched with warm water and the solids dissolved with acetonitrile. HPLC analysis shows the desired product as acid (69.1 area %−retention time 6.38 min). Excess phosphoryl trichloride was removed by distillation and careful quench of the residue with warm water. Filtration of the solid yielded a mixture of 4-cyanobenzoic acid, the terephthalic dinitrile and terephthalic acid. Alternatively, removal of the phosphoryl trichloride by distillation may be followed by vacuum distillation of the residue to isolate pure acid chloride.

1. A process for the preparation of 4-cyanobenzoyl chlorides of formula I, 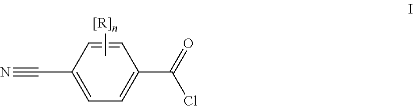
wherein
R is halogen, C₁-C₄-alkyl, C₁-C₄-haloalkyl, C₁-C₄-alkoxy, or C₁-C₄-haloalkoxy;
n 0, 1 or 2;
the process comprising reacting a compound of formula II, 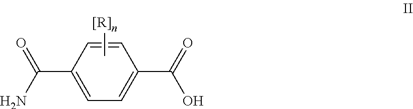
wherein the variable R is as defined above for compounds of formula I, with a chlorinating agent.

2. The process according to claim 1, wherein the chlorinating agent is selected from phosphoryl trichloride, trichlorophosphane, pentachlorophosphane, thionyl chloride, phosgene, diphosgene, triphosgene, and oxalyl chloride.

3. The process according to claim 1, wherein the chlorinating agent is phosphoryl trichloride.

4. The process according to claim 1, wherein the amount of chlorinating agent is between 2 and 15 equivalents based on the amount of compound of formula II.

5. The process according to claim 1, wherein the process is conducted in an auxiliary solvent or in the absence of an auxiliary solvent.

6. The process according to claim 5, wherein the auxiliary solvent is dioxane, tert-butyl methyl ether, di-iso-propyl ether, benzene, toluene, xylene, mesitylene, chlorobenzene, n-hexane, cyclohexane, dichloromethane, chloroform, tetrachloromethane, dichloroethane, or mixtures thereof.

7. The process according to claim 1, wherein the reaction is conducted in the absence of a solvent.

8. The process according to claim 1, wherein the reaction is conducted in the presence of substoichiometric amounts of N,N-dimethylformamide or N,N-dimethylacetamide based on the amount of compound II.

9. The process according to claim 1, wherein the reaction mixture is heated to reflux temperature or to a temperature that is within the range between the reflux temperature and a temperature that lies 50° C. below the reflux temperature.

10. The process according to claim 1, wherein the pressure is within the range between 100 kPa and 500 kPa.

11. The process according to claim 1, wherein the variable n is 1 and R in compounds of formulae I and II is fluorine.

12. The process according to claim 1, wherein the variable n is 0 in compounds of formulae I and II.

13. The process according to claim 1, further comprising reacting the compound of formula IIb, wherein the variable n is as defined for compounds of formulae I and II, 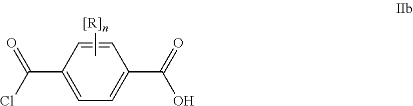
to give a compound of formula II, which is then used as defined in claim 11.

14. The process according to claim 13, further comprising reacting the compound of formula IIa, wherein the variable n is as defined for compounds of formulae I and II, in the presence of water 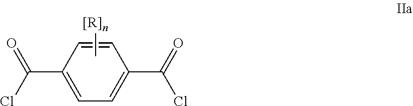
to give a compound of formula IIb, which is then used as defined in claim 12.

15. The process according to claim 12, further comprising the step of reacting the compound of formula I with an amine of formula III,
R¹—NH—R² III
wherein
R¹ is C₁-C₆-alkyl, C₁-C₆-alkoxy, C₃-C₁₁-cycloalkyl, C₃-C₈-cycloalkenyl, C₂-C₆-alkenyl, C₂-C₆-alkynyl, C₁-C₆-alkoxyimino-C₁-C₄-alkyl, C₂-C₆-alkenyloxyimino-C₁-C₄-alkyl, C₂-C₆-alkynyloxyimino-C₁-C₄-alkyl, C₁-C₆-alkylamino, diC₁-C₆-alkylamino, —C(═O)—C₁-C₆-alkyl, —C(═O)—O—C₁-C₆-alkyl, phenyl-C₁-C₄-alkyl, phenyl-C₁-C₄-alkenyl, phenyl-C₁-C₄-alkynyl, heteroaryl-C₁-C₄-alkyl, phenyl, naphthyl, or a 3- to 10-membered saturated, partially unsaturated or aromatic mono- or bicyclic heterocycle, wherein the ring member atoms of said mono- or bicyclic heterocycle include besides carbon atoms further 1, 2, 3 or 4 heteroatoms selected from N, O and S as ring member atoms with the provision that the heterocycle cannot contain 2 contiguous atoms selected from O and S; and wherein the heteroaryl group in the group heteroaryl-C₁-C₄-alkyl is a 5- or 6-membered aromatic heterocycle, wherein the ring member atoms of the heterocyclic ring include besides carbon atoms 1, 2, 3 or 4 heteroatoms selected from N, O, and S as ring member atoms with the provision that the heterocycle cannot contain 2 contiguous atoms selected from O and S; and wherein any of the above-mentioned aliphatic or cyclic groups are unsubstituted or substituted with 1, 2, 3, or up to the maximum possible number of identical or different groups R^{1a}; or
R¹ and R², together with the nitrogen atom to which they are attached, form a saturated or partially unsaturated mono- or bicyclic 3- to 10-membered heterocycle, wherein the heterocycle includes beside one nitrogen atom and one or more carbon atoms no further heteroatoms or 1, 2 or 3 further heteroatoms independently selected from N, O, and S as ring member atoms with the provision that the heterocycle cannot contain 2 contiguous atoms selected from O and S; and wherein the heterocycle is unsubstituted or substituted with 1, 2, 3, 4, or up to the maximum possible number of identical or different groups R^{1a}; wherein
R^{1a} is halogen, oxo, cyano, NO₂, OH, SH, NH₂, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, —NHSO₂—C₁-C₄-alkyl, —(C═O)—C₁-C₄-alkyl, C(═O)—C₁-C₄-alkyl, C₁-C₆-alkylsulfonyl, hydroxyC₁-C₄-alkyl, —C(═O)—NH₂, —C(═O)—NH(C₁-C₄-alkyl), C₁-C₄-alkylthio-C₁-C₄-alkyl, aminoC₁-C₄-alkyl, C₁-C₄-alkylamino-C₁-C₄-alkyl, diC₁-C₄-alkylamino-C₁-C₄-alkyl, aminocarbonyl-C₁-C₄-alkyl, or C₁-C₄-alkoxy-C₁-C₄-alkyl;
R² is hydrogen, C₁-C₆-alkyl, C₂-C₆-alkenyl, C₂-C₆-alkynyl, C₁-C₆-alkoxy, C₃-C₁₁-cycloalkyl, —C(═O)H, —C(═O)—C₁-C₆-alkyl, —C(═O)—C₃-C₁₁-cycloalkyl, or —C(═O)—O—C₁-C₆-alkyl; and wherein any of the aliphatic or cyclic groups in R² are unsubstituted or substituted with 1, 2, 3, or up to the maximum possible number of identical or different radicals selected from the group consisting of halogen, hydroxy, oxo, cyano, C₁-C₆-alkyl, C₁-C₆-alkoxy, and C₃-C₁₁-cycloalkyl;
to obtain a compound of formula IV

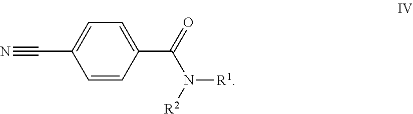

16. The process according to claim 15, further comprising reacting the compound of formula IV to obtain a compound of formula V

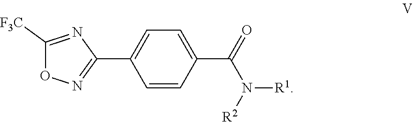

17. The process according to claim 16, further comprising the step of reacting the compound of formula V to obtain a compound of formula VI

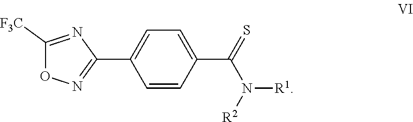

18. The process according to claim 14, wherein in compounds of formula III, IV, V and VI
R¹ is methyl, 2-methoxyiminoethyl, bicyclo[1.1.1]pentan-1-yl, 2-fluoro-phenyl, 4-fluoro-phenyl, or 2,4-difluorophenyl; in particular methyl or 2-fluoro-phenyl; and
R² is hydrogen.
